(19) United States
(12) Patent Application Publication
(10) Pub. No.: US 2005/0215857 A1
(43) Pub. Date: Sep. 29, 2005

(54) Endoscope system for efficiently operating plural signal processing apparatuses

(75) Inventor: Tomoki Iwasaki, Hachioji-shi (JP)

Correspondence Address: OSTROLENK FABER GERB & SOFFEN, 1180 AVENUE OF THE AMERICAS, NEW YORK, NY, 100368403, US

(73) Assignee: Olympus Optical Co., Ltd.

(21) Appl. No.: 10/379,864

(22) Filed: Mar. 4, 2003

(51) Int. Cl. A61B 1/04

(52) U.S. Cl. 600/118; 600/109

(57) ABSTRACT

A keyboard in an endoscope system is constituted by endoscope related information operating keys and screen control keys for controlling character input, character erasure, cursor movement and screen of monitor or filing monitor which are used as common functions and control keys used exclusively for respective outside connected apparatus in a processor or a digital file apparatus controlled thereby and the control keys include a Select key for controlling to switch the endoscope related information operating keys and the screen control keys commonly in the processor and the digital file apparatus.

### CROSS-REFERENCE

The present application is a continuation under 37 C.F.R. §1.53(b) of prior application Ser. No. 09/573,855, filed May 18, 2000, by Tomoki IWASAKI entitled ENDOSCOPE SYSTEM FOR EFFICIENTLY OPERATING PLURAL SIGNAL PROCESSING APPARATUSES.

This application claims benefit of Japanese Application No. Hei 11-142145 filed in Japan on May 21, 1999, the contents of which are incorporated by this reference.

### BACKGROUND OF THE INVENTION

1. Field of the Invention

The present invention relates to an endoscope system, and particularly to an endoscope system having a control unit for controlling a plurality of signal processing apparatus.

2. Related Art Statement

In recent years, an apparatus having an operating means for controlling a plurality of apparatus has been used.

In Japanese Patent Laid-Open No. 198117/1991, for example, an operating apparatus for operating a display unit is shown, in which an operating unit input control includes a keyboard and an electronic touch panel having a switch, the switch being capable of selectively connecting the touch panel and the keyboard to the work station exclusively.

Japanese Patent Laid-Open No. 124174/1995, for example, shows a medical control system that has an exclusive concentrated setting control panel, the system being thus capable of carrying out integrally concentrated control of a plurality of medical apparatus by connecting the panel with the medical apparatus by a communication cable.

Further, in an endoscope system for taking an image of the inside of the inside of the coelom, the printing of an image and the endoscope related data on a number of print sheets and a number of divided sheets is well known, and is conventionally carried out by making appropriate settings at the printer.

However, in Japanese Patent Laid-Open No. 198117/1991 (mentioned above) the operating unit is divided by respective functions (keyboard and electronic touch panel), which makes the unit inefficient and large.

Further, in Japanese Patent Laid-Open No. 124174/1995 (mentioned above) the exclusive concentrated setting control panel results in the apparatus becoming large and expensive.

Further, in the conventional endoscope system (described above), when images or endoscope related data are printed, the number of print sheets, and the number of divided sheets, etc., are set at the printer. This makes operating the endoscope system inefficient since the printer is operated instead of a processor.

### OBJECTS AND SUMMARY OF THE INVENTION

It is an object of the invention to provide an endoscope system capable of efficiently operating a plurality of signal processing apparatus simply and compactly.

According to an embodiment of the invention, an endoscope is disclosed which include an endoscope, a plurality of endoscope related information apparatus for processing endoscope related information, an inputting means to execute an inputting operation to control the endoscope and the plurality of endoscope related information apparatus, a selectively inputting means for selection of an apparatus that is controlled by the inputting means such as the endoscope and the plurality of endoscope related information apparatus, and a system controlling means that upon receiving a signal from the selectively inputting means selects the apparatus controlled by the inputting means.

Other characteristics and advantages of the invention will become sufficiently apparent from the following detailed description.

### BRIEF DESCRIPTION OF THE DRAWINGS

FIG. 1 through FIG. 9 relate to an endoscope system according to a first embodiment of the invention:

FIG. 1 is a constitution diagram showing an embodiment of an endoscope system;

FIG. 2 shows an embodiment of a keyboard that may be a part in the system of FIG. 1;

FIG. 3 is a constitution view showing a set of keys for controlling a printer;

FIG. 4 is a first flowchart of an operation of the endoscope system of FIG. 1;

FIG. 5 is a first example of a display provided by a monitor after a processing according to FIG. 4;

FIG. 6 is a second example of a display provided by the monitor after a processing according to FIG. 4;

FIG. 7 is a second flowchart of an operation of the endoscope system of FIG. 1;

FIG. 8 is an example that is printed by the printer after a processing according to FIG. 7;

FIG. 9 is a constitution view showing a modified example of the keyboard of FIG. 2.

FIG. 10 and FIG. 11 relate to an endoscope system according to a second embodiment of the invention:

FIG. 10 is a constitution view showing an embodiment of an operation panel;

FIG. 11 is a flowchart of an operation of an endoscope system having the operation panel of FIG. 10.

FIG. 12 through FIG. 14 relate to a third embodiment of the invention:

FIG. 12 is a constitution diagram showing an embodiment of an endoscope system, FIG. 13 is a flowchart of an operation of the endoscope system of FIG. 12; FIG. 14 is an example of a display provided by a monitor after a processing according to FIG. 13.

### DETAILED DESCRIPTION OF THE PREFERRED EMBODIMENTS

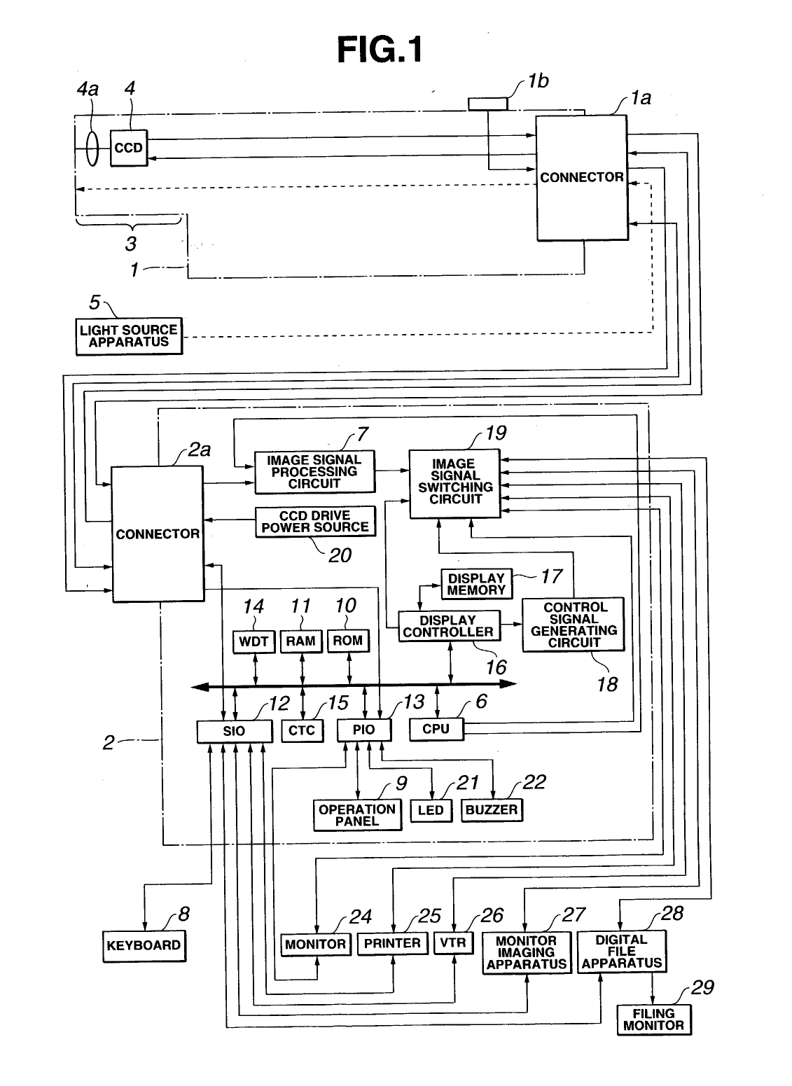

As shown by FIG. 1, an endoscope system according to a first embodiment includes: an endoscope 1 for taking image from inside of the coelom, a possessor 2 for subjecting an image signal taken by the endoscope 1 to signal processing, and a light source apparatus 5 for supplying irradiation light to the endoscope. The light source apparatus 5 is connected to the endoscope 1, via a connector la of the endoscope 1 and the processor 2 is connected to the endoscope 1 via a connector 2a and the connector 1a.

The endoscope 1 includes an object lens 4a and a CCD (solid-state imaging element) 4 arranged at a distal end of an inserting portion 3 for taking an image of a subject and converting the image into an electric signal, a release switch 1b, a light guide (not explicitly shown) and so on. The irradiation light supplied from a lamp of the light source apparatus 5 is transferred by the light guide and is emitted onto the subject from a distal end portion attached to an irradiation window (not explicitly shown) provided at the distal end portion of the inserting portion 3.

The processor 2 includes a CPU 6 for executing a plurality of calculation processings; an image signal processing circuit 7 controlled by CPU 6 for executing image processing of an image signal photoelectrically converted from CCD 4 in the endoscope 1; a rewritable involatile ROM 10 for storing and holding programs for operating CPU 6, user information set by a keyboard 8, an operation panel 9, and the like; a calculation memory RAM 11 used for calculation or the like; a serial communication controller (SIO) 12 communicating via a serial interface and transmitting and receiving control command or a plurality of data after serial conversion to and from outside connected apparatus (described later); a parallel communication controller (PIO) 13 communicating via a parallel interface; a watchdog timer (WDT) 14 for preventing run away of CPU 6; a counter timer (CTC) 15; a display controller 16 for controlling the display of endoscope image subjected to image processing by the image signal processing circuit 7, and information related to the endoscope image such as patient data, date and hour, comment and so on (hereinafter, referred to as endoscope related information); a display memory 17 which is a memory for the display controller; an image signal switching circuit 19 for switching the endoscope image from the image signal processing circuit 7, display data of the endoscope related information outputted from the display controller 16 and input and output signals to and from the outside connected apparatus (described later) by a control signal generating circuit 18 and CPU 6; and a CCD drive power source 20 for driving CCD 4, the operation panel 9 for operating the processor 2, LED 21, a buzzer 22 and the like.

The processor 2 controls CPU 6 by programs stored in ROM 10 and executes the following operations:
Control of LED 21 and the buzzer 22 by way of PIO 13;
Formation of the endoscope related information data by the display controller 16 and the display memory 17;
Outputting an image of the endoscope 1 and the endoscope related information to a monitor 24, a printer 25, VTR 26, a monitor imaging apparatus 27 and a digital file apparatus 28 by the display controller 16, the display memory 17, the control signal generating circuit 18 and the image signal switching circuit 19;
Image processing of the endoscope image by the image signal processing circuit 7; and
Control of the monitor 24, the printer 25, VTR 26, the monitor imaging apparatus 27 and the digital file apparatus 28 by way of SIO 12 or PIO 13.

The above-described operations are controlled also by controlling CPU 6 by the operation panel 9 and the keyboard 8.

The processor 2 is connected to a plurality of outside connected apparatus (e.g., keyboard 8, monitor 24, printer 25, VTR 26, monitor imaging apparatus 27, digital file apparatus 28). The processor 2 and the plurality of outside connected apparatus can communicate endoscope related information as endoscope related information apparatuses.

The monitor 24 displays the endoscope image, and the endoscope related information generated by the image signal processing circuit 7 and the display controller 16, and the image inputted from the printer 25, VTR 26, the monitor imaging apparatus 27 and the digital file apparatus 28 to the processor 2 by switching image to be displayed by the image signal switching circuit 19.

The printer 25 stores a plurality of images switched and inputted by the image signal switching circuit 19 and prints the endoscope image in accordance with setting of a number of endoscope images printed by the printer 25 on one sheet of print paper (hereinafter, referred to as division number) and a number of print sheets printed by one printing operation.

Further, by a release signal inputted by the release switch 1b in the endoscope 1 and inputted to CPU 6 via PIO 13, CPU 6 transmits the release signal to the printer 25, VTR 26, the monitor imaging apparatus 27 and the digital file apparatus 28 via SIO 12 and records dynamic image or stationary image outputted from the image switching circuit 7.

The digital file apparatus 28 includes an image compression apparatus and an image control apparatus (including personal computer) (not explicitly shown) and can be displayed by a filing monitor 29 instead of monitor 24.

The keyboard 8 is connected to the processor 2 via SIO 12. The keyboard 8 is provided with operation keys for inputting or operating the endoscope related information and control keys for controlling the outside connected apparatus to transmit and receive command or data.

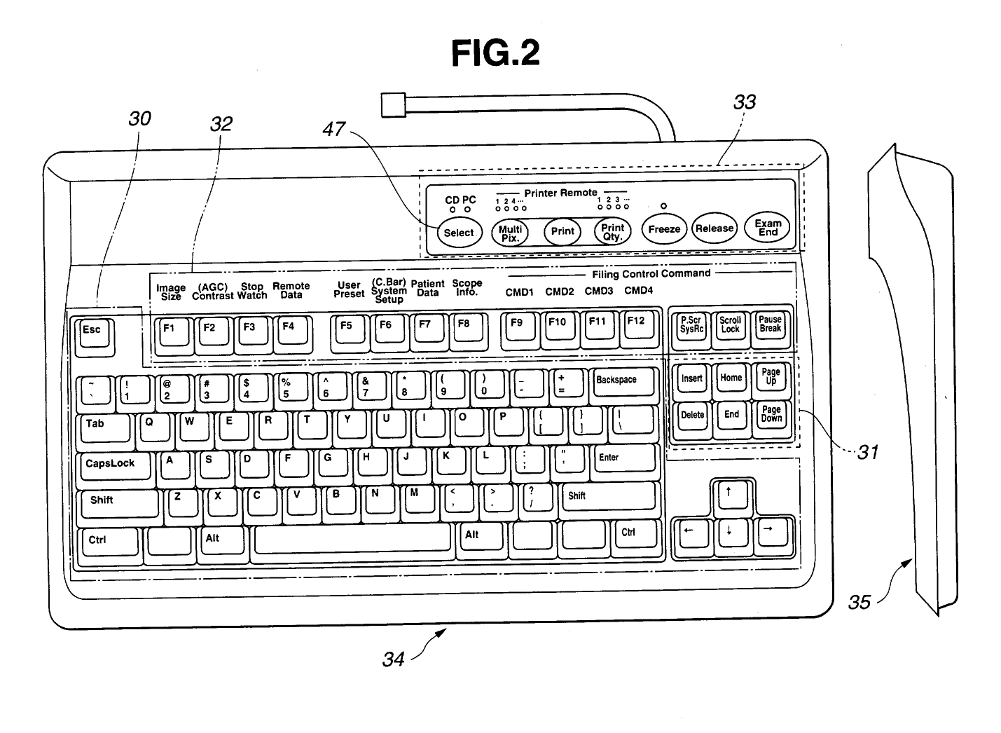

FIG. 2 shows the keyboard 8 in detail. Notation 34 designates an upper view of the keyboard 22 and notation 35 designates a side view of the keyboard 8.

Keys 30 and 31 designate endoscope related information operation keys and screen control keys of character input, character erasure, cursor movement, control of screen of the monitor 24 or the filing monitor 29 and the like which are used as common functions in the processor 2 and the digital file apparatus 28 controlled by the keyboard 8. By inputting respective key switches, command or data corresponding to the keys is transmitted and received.

Keys 30 on the keyboard 8 conform with, for example, Japanese Industrial Standards (JIS) or ASCII, input characters and signs printed on the keys and execute various related operation accompanied by character input such as character erasure, character selection or the like.

Keys 32 and 33 designate control keys used exclusively for respective outside connected apparatus. By inputting respective keys, command or data for controlling respective outside apparatus is transmitted and received.

Further, the keys 30 through 32 are mechanical switches and accordingly, the keys are provided with stroke and is excellent operability.

Further, the keys 30 through 32 may execute endoscope related operation or screen control other than respective key functions printed at the keys or upper portions of the keys when a plurality of keys (for example, Ctrl+“C”) are simultaneously depressed.

Further, print portions of the keys 32 for indicating respective key functions may be at the key top portions of the keys (upper side portions of the keys per se) or may be at surrounding portions of the keys (for example, upper sides of the keys as shown by FIG. 2). Respective functions of the keys 32 are shown by following Table 1.

TABLE 1

| Name | Controlling | | |
|---|---|---|---|
| of key | apparatus | Function | |
| F1 | Processor | ImageSize | At each depression of key |
| | | | endoscope image size is |
| | | | switched. |
| F2 | | Contrast | At each depression of key |
| | | | contrast mode previously |
| | | | preset by processor is |
| | | | switched. |
| | | (AGC) | While depressing shift key at |
| | | | each depression of key ON/OFF |
| | | | of AGC (auto gain control: |
| | | | automatic control of brightness |
| | | | of endoscope image on monitor) |
| | | | is switched. |
| F3 | | Stopwatch | At each depression of key |
| | | | stopwatch data displayed on |
| | | | monitor is switched as |
| | | | start→stop→reset. When |
| | | | key is depressed while depressing |
| | | | shift key in stopping stopwatch, |
| | | | stopwatch is restarted. |
| F4 | | RemoveData | At each depression of key |
| | | | display/nondisplay of endoscope |
| | | | related information displayed on |
| | | | monitor is switched. |
| F5 | | UserPreset | At each depression of key set |
| | | | screen for registering plural |
| | | | system sets corresponding to |
| | | | plural users is |
| | | | displayed/nondisplayed. |
| F6 | | SystemSetup | At each depression of key user |
| | | | set screen related to control |
| | | | of processor and peripheral |
| | | | apparatus is |
| | | | displayed/nondisplayed. |
| | | (C. Bar) | At each depression of key |
| | | | while depressing shift key |
| | | | display/nondisplay of color |
| | | | bar on monitor is switched. |
| F7 | | PatientData | At each depression of key |
| | | | patient data set screen for |
| | | | call/register set of patient |
| | | | data stored in ROM or RAM in |
| | | | processor is |
| | | | displayed/nondisplayed. |
| F8 | | Scopelnfo | At each depression of key |
| | | | endoscope related information |
| | | | read from storing unit stored |
| | | | in scope by processor is |
| | | | displayed/nondisplayed. |
| F9 | Digital file | CMD 1 | Command for controlling digital |
| | | | file apparatus by depressing key |
| F10 | apparatus | CMD 2 | |
| F11 | | CMD 3 | |
| F12 | | CMD 4 | |
| P. ScrS | Digital file | CMD 5 | Command for controlling digital |
| SysRq | apparatus | | file apparatus by depressing key |
| Scroll | | CMD 6 | |
| Lock | | | |
| Pause | | CMD 7 | |
| Break | | | |

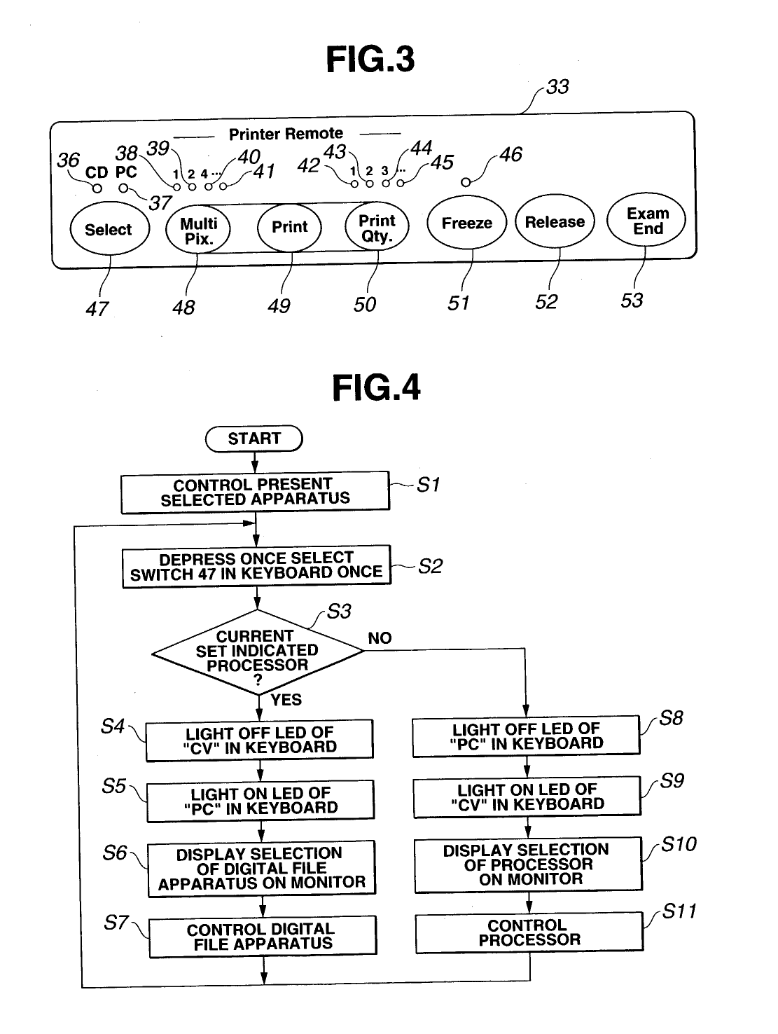

The keys 33 include a plurality of flat keys 47 through 53 and a plurality of LED 36 through 46 as shown by FIG. 3. The flat keys can be wiped easily and are water proof and chemical proof. Accordingly, they can be sterilized easily. Further, they can be fabricated more inexpensively than mechanical switches. Functions of the keys 33 and LED are shown by Table 2 and Table 3.

TABLE 2

| | Controlling | |
|---|---|---|
| Key | apparatus | Function |
| Select | Processor | At each depression of switch apparatus |
| | or digital | controlled by keyboard is switched. |
| | file | LED indicates controlling apparatus and |
| | apparatus | is alternately lighted at each depression |
| | | of switch. |
| | | In lighting “CV”, processor is controlled. |
| | | In lighting “PC”, digital file apparatus is |
| | | controlled. |
| MultiPix. | Processor | At each depression of switch division |
| | or | number of image of 1 sheet of print paper |
| | printer | printed from printer is switched. LED |
| | | indicates number of division and is |
| | | lighted one by one at each depression of |
| | | switch as follows. |
| | | 1→2→4→ . . . |
| | | 1: (LED 38) 1 division |
| | | 2: (LED 39) 2 division |
| | | 4: (LED 40) 4 division |
| | | . . . : (LED 41) division number set by |
| | | user set |
| | | (8 or 9 or 16 division) |
| Print | Processor | Print endoscope image displayed on monitor |
| | or | is printed by sheet number set by |
| | printer | PrintQty. switch. |
| PrintQty. | Processor | At each depression of switch, sheet number |
| | or | of print paper printed is switched. LED |
| | printer | designates printed sheet number and LED |
| | | is lighted at each depression of switch |
| | | as follows. |
| | | □1→2→3→ . . . |
| | | 1: (LED 42) 1 sheet |
| | | 2: (LED 43) 2 sheet |
| | | 3: (LED 44) 3 sheet |
| | | . . . : (LED 45) sheet number set by user |
| | | set (4˜9 sheet) |

Further, an upper face of (face of depressing keys) the keyboard 8 may be covered with a sheet to make the keyboard 8 waterproof and dust proof. Control may then be carried out by depressing the respective keys of the keyboard 8 from above the sheet.

Next, operation of the embodiment will be explained.

FIG. 4 is a flowchart showing the switching of the processor 2 or the digital file apparatus 28 by the Select key 47 of the keyboard 8 (refer to FIG. 3).

First, as shown by FIG. 4, at step S1, after the power supply of the processor 2 is turned ON, the apparatus that is preset by a program stored in ROM 10 (for example, processor 2) is controlled. Specifically, firstly, a command is transmitted from CPU 6 to the keyboard 8 via SIO 12 and LED 36 that is lighted in selecting the processor 2 (refer to FIG. 3) is lighted to display that the keyboard 8 selects and controls the processor 2.

When processor 2 is selected, firstly, by the keys 30 and 31, endoscope related information operation and screen control of character code input, character erasure, cursor movement, screen control (monitor 24) and so on, which are used as common functions, are carried out. Specifically, by depressing respective keys of the keys 30 and 31, command or data in correspondence with the keys is inputted to CPU 6 via SIO 12.

Receiving command or data, CPU 6 controls the processor 2 to perform the following.
Formation of endoscope related information data displayed by the display controller 16 and the display memory 17;
Outputting an image taken by the endoscope 1, and the endoscope related information to the monitor 24, the printer 25, VTR 26, the monitor imaging apparatus 27 and the digital file apparatus 28 by the display controller 16, the display memory 17, the control signal generating circuit 18 and the image signal switching circuit 19; and
Image processing of the endoscope image by the image signal processing circuit 7.

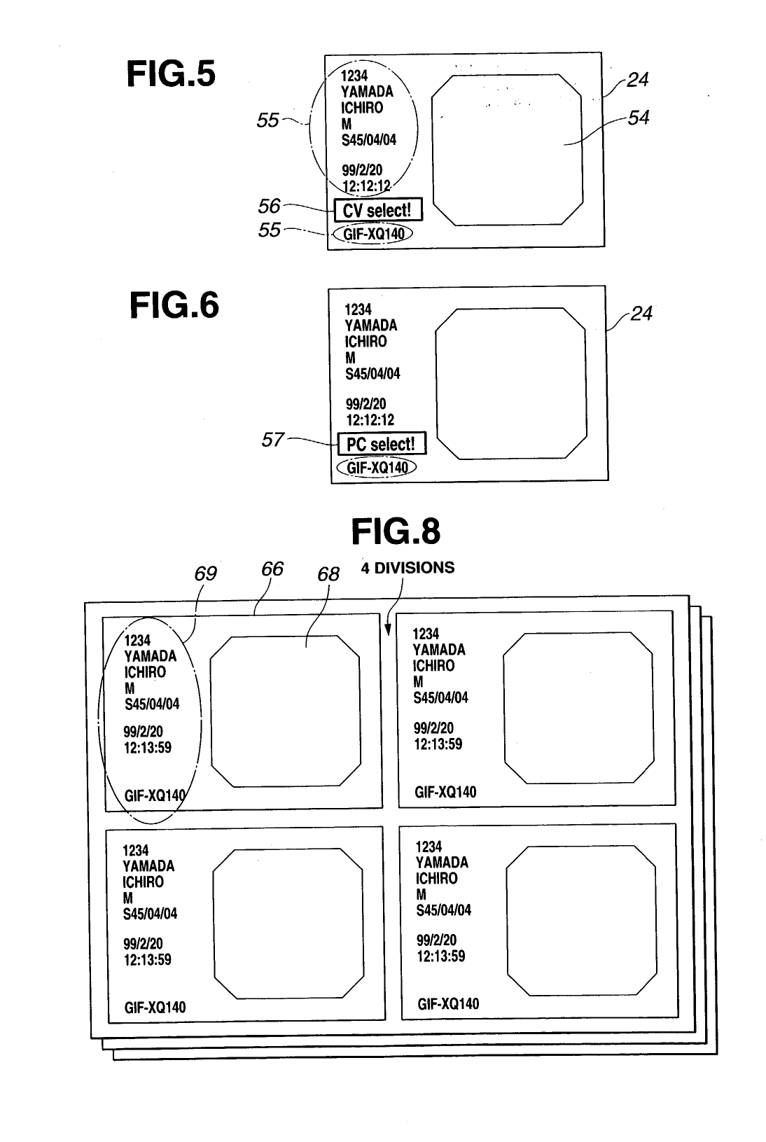

On the monitor 24, as shown by FIG. 5, “CV select!” 56, which is a display indicating that the processor 2 is selected, may be displayed on the screen comprising an endoscope image displaying portion 54 and patient data 55. Further, the display may be erased automatically after a pre-selected time period or may be erased by key input in the operation panel 9 or the keyboard 8.

Meanwhile, the keys 32 and 33 in the keyboard 8 are controlled by CPU 6 such that control keys of the processor 2 the apparatus exclusive keys of the apparatus (shown above) and control keys of the printer 25 (total keys in keys 33 and F1 through F8 in keys 32) function, and other control keys do not function.

Next, at step S2, when the Select key 47 in the keyboard 8 (refer to FIG. 3) is depressed once, command transmitted from the keyboard 8 is inputted into the processor 2 via SIO 12 and at step S3, after receiving a command, CPU 6 firstly confirms currently selected apparatus.

When the currently selected apparatus is the processor 2, CPU 6 transmits a command via SIO 12, at step S4, turns off LED 36 for selecting processor (refer to FIG. 3) and, at step S5, turns on LED 37 for selecting the digital file apparatus (refer to FIG. 3) to indicate that the digital file apparatus 28 is selected.

Further, at step S6, on the monitor 24, as shown by FIG. 6, “PC select!” 57 is displayed, which indicates that the digital file apparatus 28 is selected. The display may be erased automatically after a pre-selected time period or may be erased by input of keys in the operation panel 9 or the keyboard 8.

When digital file apparatus 28 is selected, at step S7, firstly, by the keys 30 and 31 endoscope related information operation and screen control of character code input, character erasure, cursor movement, screen control (filing monitor 29) and the like, which are used as common functions, are carried out. Specifically, command or data inputted from the keys 30 and 31 in the keyboard 8 is inputted to CPU 6 via SIO 12.

After receiving a command or data, CPU 6 transmits the command to the digital file apparatus 28 via SIO 12. After receiving the command, the digital file apparatus 28 controls the operation of the filing monitor 29 from the command.

At this stage, the processor 2 only transmits command or data transmitted from the keyboard 8 to the digital file apparatus 28 and does not execute control of the processor 2 per se by a command or data from the keyboard 8 (except control by Select key 47).

Meanwhile, CPU 6 controls the keys 32 and 33 in the keyboard 8 such that control keys of the digital file apparatus 28 (only P.Scr/SysRq, ScrollLock, Pause/Break in key 32) function and other control keys do not function.

When the currently selected apparatus is the digital file apparatus 28, the operation proceeds from step S3 to step S8, CPU 6 transmits command via SIO 12 and at step S8, LED 37 (refer to FIG. 3) is turned off, at step S9, LED 36 (refer to FIG. 3) is turned on to thereby indicate that the processor 2 is selected.

Further, on the monitor 24, as shown by FIG. 5, at step S10, there is displayed “CV select!” 56, which is a display indicating that the processor 2 is selected. The display may be erased automatically after a pre-selected time period or may be erased by key input in the keyboard 8.

Thereafter, (when processor 2 is selected), at step S11, as described above, by the keys 30 and 31 of the keyboard 8, endoscope related information operation and screen control of character code input, character erasure, cursor movement, screen control (monitor 24) and the like, which are used as common functions are executed. CPU 6 controls the keys 32 and 33 in the keyboard 8 such that only control keys of the processor 2 by apparatus exclusive keys (shown above) and control keys of the printer 24 (total keys in keys 33 and F1 through F8 of keys 32) function, and other control keys do not function. Command or data transmitted from the keyboard 8 is not transmitted as it is to the digital file apparatus 28 via SIO 12.

After the processing of step S7 or step S11, the operation returns to step S2 and every time the Select key 47 is depressed, processing for the switching of the processor 2 or the digital file apparatus 28 is repeated.

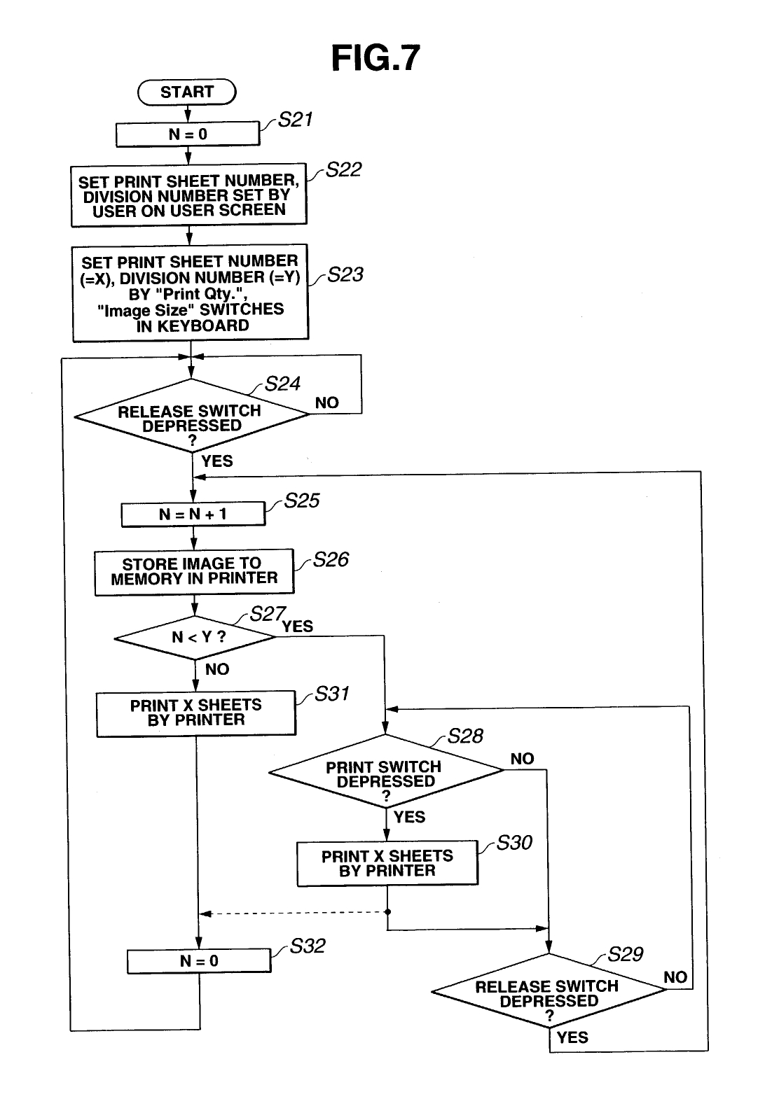

FIG. 7 is a flowchart showing details of control of the printer 25 by the printer control keys 48 through 50 in the keyboard 22.

Once the power is turned ON, at step S21, the number of sheets of image (N) stored in the printer 25 is set to 0 sheet. Thereafter, at step S22, by depressing the SystemSetup key (F6) in the keys 32, or by depressing a system set key (not explicitly shown) on the operation panel 9, a system set screen is displayed on the monitor 24. On the system set screen, a user sets division number (which becomes effective when LED 41 is on: refer to FIG. 3) and a number of print sheets (which becomes effective when LED 45 is on: refer to FIG. 3) which are set by the user per se. The user pertinently selects the division number from 8/9/16 and the print sheet number from 4 through 9 sheets.

Thereafter, at step S23, by depressing the MultiPix. key 48 on the keyboard 8, the division number in printing by printer (for example, in the case of 4 divisions, up to 4 endoscope images are printed on 1 sheet of print paper) is set, and by depressing the PrintQty key 50, a number of sheets (for example, 5 sheets) for printing out endoscope image stored in the memory of the printer is set (refer to FIG. 3).

Thereafter, at step S24, by inputting the release signal by the release switch 1b in the endoscope 1 via PIO 13 or by inputting the release signal by the Release key 52 on the keyboard 8 (refer to FIG. 3) via SIO 12, at step S25, the image sheet number N stored in the memory is added with +1 and at step S26, the release signal is transmitted to the printer 25 by CPU 6 via SIO 12 and the printer 25 inputs 1 image of the image transmitted from the image signal switching circuit 19 to the inside memory (not explicitly shown).

Next, at step S27, the division number Y set at step S23 is compared with the image sheet number N stored in the memory by the release operation.

When N<Y at step S27, at step S28, it is determined whether the Print key 49 (refer to FIG. 3) on the keyboard 8 is depressed and when the Print key 49 is not depressed, at step S29, it is determined whether the release switch 1b is depressed.

When the Print key 49 is depressed at step S28, at step S30, the number of sheets (X) set at step S23 are printed out and the operation is on standby until the release switch 1b in the endoscope 1 or the Release key 52 on the keyboard 8 is depressed (step S28 or step S29). When the Print key 49 is not depressed and the Release key 52 is depressed, the situation is fed back to the process of step S25 and the value of N is increased by 1.

When N>Y in step S27, at step S31, the number of sheets (X sheets) set at step S23 are printed and at step S32, N is cleared and the operation returns to step S24.

FIG. 8 shows print paper printed by the division number (4 divisions in this case) and the print sheet number (3 sheets in this case) set at step S23 of FIG. 7. Notation 66 designates 1 image inputted to the printer 24, notation 68 designates endoscope image taken by the endoscope 1 and notation 69 designates endoscope related information. Setting the division number and setting the sheet number are carried out by respectively depressing the MultiPix. key 48 and the PrintQty. key 50 (refer to FIG. 3).

In this way, by using printer exclusive keys (48 through 50) on the keyboard 8, control of the printer 25 can easily be carried out and accordingly, a user can efficiently print the endoscope image at request.

Further, according to the embodiment, after depressing the Print key 49 of the keyboard 8 (step S28, step S30), as shown by a broken line, the operation may proceed to the processor of step S32, initialize image in the memory in the printer 25 and the release time number N (N=0) and proceed to the processor of step S24.

Further, after the image sheet number becomes N=0 at step S32, setting at step S22 and step S23 may always be carried out, or may be omitted by holding set values at the preceding time or carried out in accordance with the necessity of the user.

Further, when reaching the process of step S32, the memory in the printer 25 may be cleared immediately (N=0) or the image sheet number N may be not set to N=0 and new endoscope image may be stored to the memory by overwriting thereof when the processing reaches step S26 again.

Further, a plurality of memories may be provided or a plurality of data storing areas may be provided in a single memory and a destination for holding an endoscope image may be switched.

Further, although in this embodiment the division number (LED 41) is set by the user to be 8, 9 or 16 and the print sheet number (LED 45) is set by the user to be 4 through 9 sheets, the division number and the print sheet number are not limited to the numerical values but may be set to other numerical values.

Further, the numerical values of the division number and the print sheet number may be pre-selected and provided by the endoscope system or may freely be set by the user.

Further, in clearing endoscope related character information by depressing the ExamEnd switch 53 (refer to FIG. 3), the information may be cleared without having to print an unprinted endoscope image held in the memory of the printer 25, and the user may designate whether the information is to be cleared or not.

Further, the Print key 49 on the keyboard 8 may be provided on the operation panel 9 of the processor 2 instead of being provided on the keyboard 8.

Further, the Print key 49 may not be limited only to the print key of image but, for example, where there are a plurality of memories for storing images in the printer 25 or there are a plurality of image storing areas in the memory, the Print key 49 may be used as a switch for switching the memories or the memory areas.

As explained above, according to the embodiment, by switching using the Select key 47, the keys 30 and 31 can commonly be used in the processor 2 and the digital file apparatus 28. Accordingly, a single keyboard can operate both apparatus and accordingly, operation is more efficient and the size of the system is reduced. Further, the keys 32 and 33 are restricted to use only for operating the apparatus selected by the Select key 47 and accordingly, erroneous operation due to erroneous input of keys can be prevented.

Further, although according to the embodiment, only the processor 2 and the digital file apparatus 28 are controlled to select by the Select key 47, the printer 25, VTR 26 and the monitor imaging apparatus 27, which are other outside apparatus, also may be selected and controlled.

Further, other than preparing a plurality of keys 32 and 33 for each outside apparatus, to reduce the size of the system, same keys may be commonly used and the apparatus may be switched by the Select key 47.

Further, portions of the keys 30 and 31 may be control keys used exclusively for the operation of an outside apparatus. In that case, to reduce the size of the system, same keys may be commonly used or may be switched by the Select key 47.

Further, the keys 32 and 33 may control the operation of a respective outside apparatus without depending on the Select key 47. In such a case, the respective outside connected apparatus can be controlled without selecting the apparatus by depressing the Select key 47 and accordingly, the outside connected apparatus can be controlled simply and instantaneously.

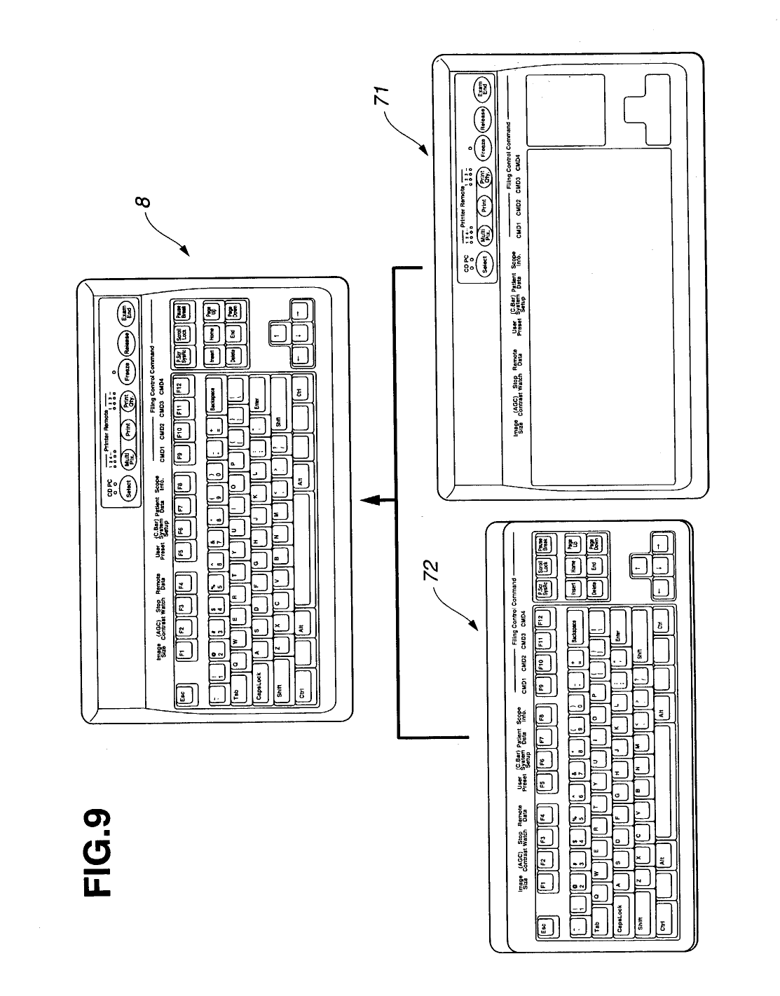

Further, as shown by FIG. 9, the key board 8 may include a cover case 71 provided with keys 33, circuits for controlling keys 33 and printed portions indicating functions of keys 32 and a keyboard main body 72 provided with keys 30 through 32. An integrated keyboard can be made when the cover case 71 and the keyboard main body 72 can be separated from each other and the keyboard 8 is constructed by covering the cover case 71 on the keyboard main body 72 to connect them, and cables of the keyboard main body 72 are stored in the keyboard 8. Thereby, expansion of function and efficient operation can be achieved.

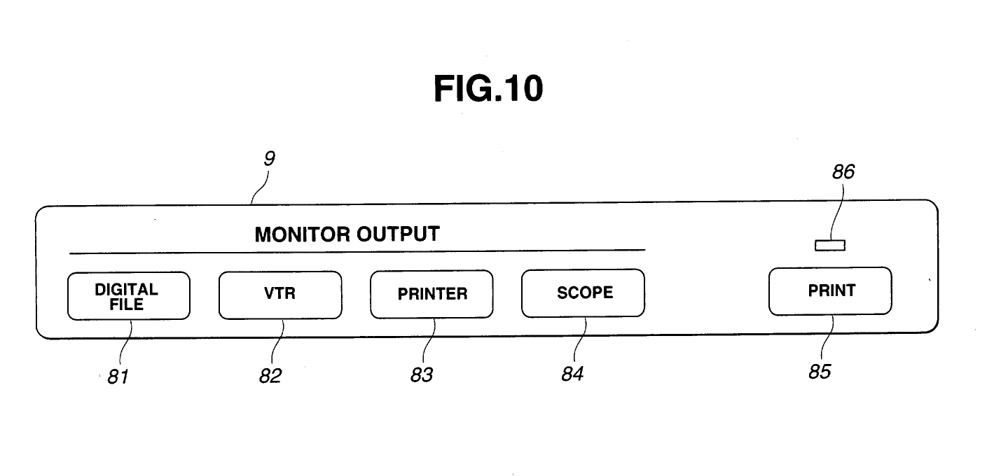

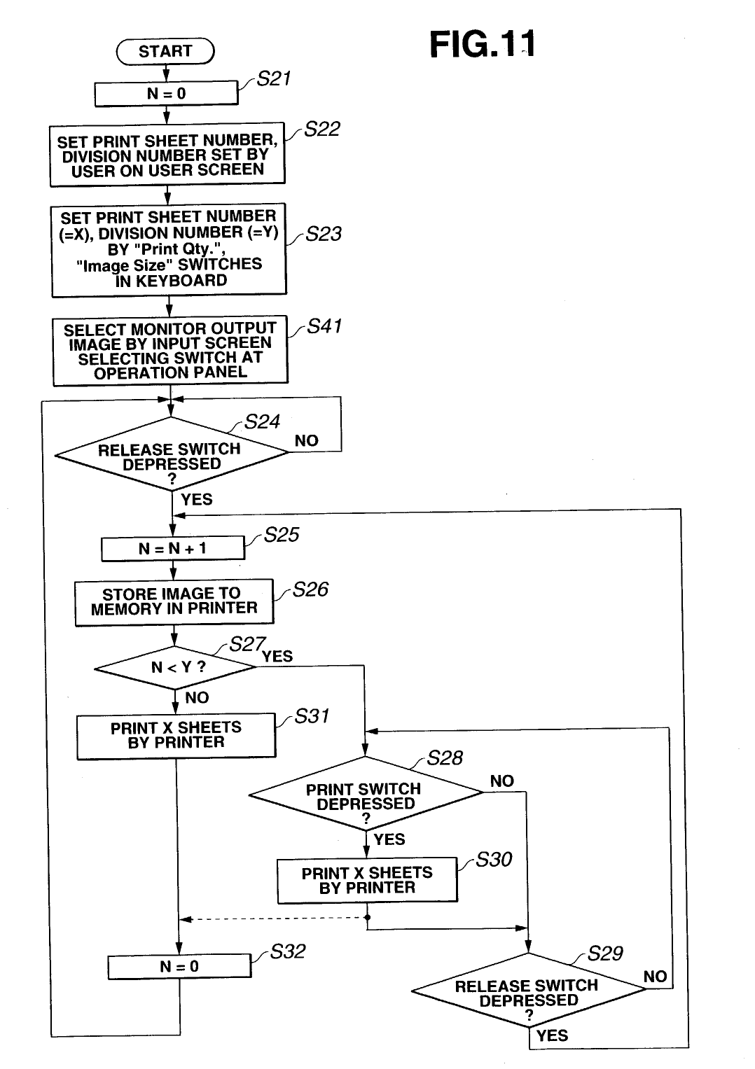

FIG. 10 and FIG. 11 relate to a second embodiment of the invention, FIG. 10 showings an operation panel and FIG. 11 is a flowchart of the operation of an endoscope system having the operation panel of FIG. 10.

The second embodiment is almost the same as the first embodiment and accordingly, an explanation will be given of only points different from the first embodiment.

In this embodiment the printer 25 is controlled by switches in the operation panel 9 of the processor 2 and, other than key portions and control circuits of the operation panel 9, this embodiment includes the same parts as that of the first embodiment.

FIG. 10 shows the operation panel 9 of the processor 2. A digital file switch 81, a VTR switch 82, a printer switch 83 and a scope switch 84 are switches for selecting an image outputted to the monitor 24 and the printer 25, and a print switch 85 is a switch for instructing print to the printer 25.

When the digital file switch 81 is depressed, a signal is inputted to CPU 6 via PIO 13 and CPU 6 outputs the image inputted from the digital file apparatus 28 to the monitor 24, the printer 25, VTR 26 and the monitor imaging apparatus 27 by controlling the display controller 16, the image signal generating circuit 18 and the image signal switching circuit 19.

When the VTR switch 82 is depressed, a signal is inputted to CPU 6 via PIO 13 and CPU 6 outputs the image inputted from VTR 26 to the monitor 24, the printer 25, the monitor imaging apparatus 27 and the digital file apparatus 28 by controlling the display controller 16, the control signal generating circuit 18 and the image signal switching circuit 19.

When the printer switch 83 is depressed, a signal is inputted to CPU 6 via PIO 13 and CPU 6 outputs the image inputted from the printer 25 to the monitor 24, VTR 26, the monitor imaging apparatus 27 and the digital file apparatus 28 by controlling the display controller 16, the control signal generating circuit 18 and the image signal switching circuit 19.

When the scope switch 84 is depressed, a signal is inputted to CPU 6 via PIO 13 and CPU 6 outputs the image inputted from the endoscope 1 and the endoscope related information to the monitor 24, the printer 25, VTR 26, the monitor imaging apparatus 27 and the digital file apparatus 28 by controlling the display controller 16, the control signal generating circuit 18 and the image signal setting circuit 19.

According to the print switch 85, similar to the release switch 1 b in the endoscope 1, every time the switch is depressed, the release signal is inputted to CPU 6 via PIO 13, the release signal is inputted to the printer 25 from CPU 6 via PIO 13 and 1 the image is stored to the memory inside of the printer 25. In FIG. 10, notation 86 designates LED which may be lighted for a constant time period or winked when the print switch 85 is depressed or may be lighted for a constant time period or winked during a time period in which the printer 25 is printing print paper, as mentioned above.

FIG. 11 is a flowchart showing details of control of the printer 25 by the printer control keys 48 through 50 and the keys of the operation panel 9 according to the embodiment.

As shown by FIG. 11, according to the embodiment, in the flowchart showing details of control of the printer 25 by the printer control keys 48 through 50 in the keyboard 22 explained in reference to FIG. 7, a step S41 by the operation panel 9 of the embodiment is added between step S23 and step S24 and other processes are the same as those in the first embodiment.

According to the added step S41, by depressing the digital file switch 81, VTR switch 82, the printer switch 83 or the scope switch 84, which is a monitor output selecting key in the operation panel 9, the input image to the processor 2 and the output image to the monitor 24 are selected and the processing proceeds to step S24.

This way, by controlling the selection of the monitor output by the digital file switch 81, VTR switch 82, the printer switch 83 and the scope switch 84 on the operation panel 9, endoscope image provided by the selected peripheral apparatus can be easily printed.

Further, according to input/output control of the processor 2 by the digital file switch 81, VTR switch 82, the printer switch 83 and the scope switch 84 of the operation panel 9 in step S41, the set state may be held by storing the state to ROM 10 or RAM 11 after it has been set once, or it may be held by turning power supply OFF→ON.

Further, the Print key 49 in the keyboard 8 may output the release signal and store the image in the memory in the printer 25 similar to the print switch 85 in the operation panel 9 instead of printing the image forcibly. Conversely, the print switch 85 in the operation panel 9 may carry out printing forcibly similar to the Print key 49 in the keyboard 8 instead of outputting the release signal.

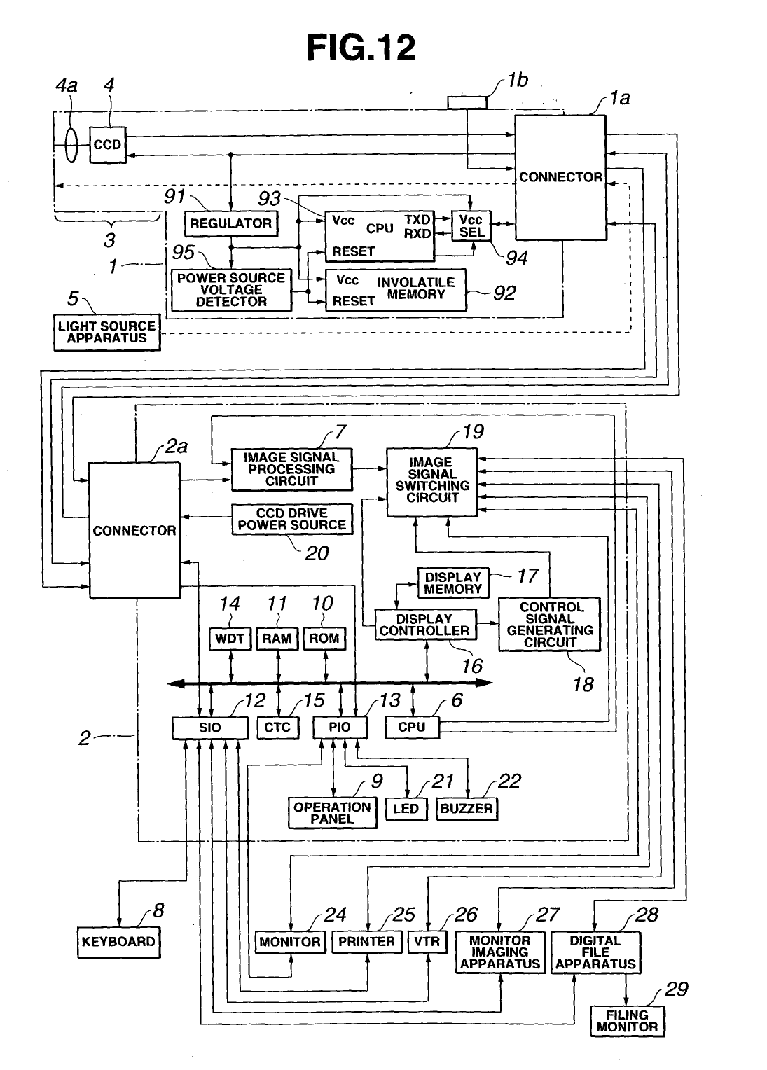

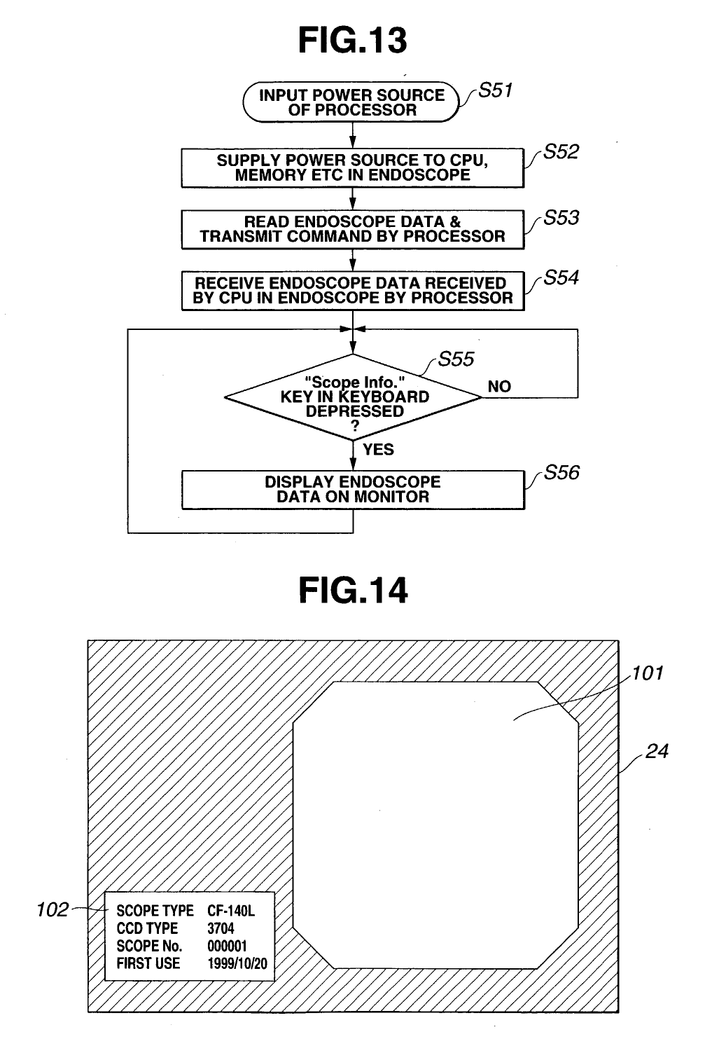

FIG. 12 through FIG. 14 relate to a third embodiment. FIG. 12 is an endoscope system, FIG. 13 is a flowchart of the operation of the endoscope system of FIG. 12, and FIG. 14 is an example of a display provided by a monitor according to processing of FIG. 13.

The third embodiment is almost the same as the first embodiment and accordingly, an explanation will be given of only of points from the first embodiment.

As shown in FIG. 12, according to the embodiment, the endoscope 1 includes the object lens 4a; the CCD 4 arranged at the distal end of the inserting portion 3 for taking an image of a subject and converting the image into an electric signal; a regulator 91 for converting the CCD drive voltage supplied from the CCD drive power source 20 in the processor 2 into a desired voltage; a rewritable involatile memory 92 of EEPROM or flash ROM or the like, storing and holding the type of the endoscope 1, the type of the CCD 4 and a plurality of data related to the endoscope 1 such as accumulated number of electricity conduction and the like; CPU 93 for carrying out a plurality of calculation processings such as the processing of accumulating the number of electricity conduction; a serial communication processing for transmitting and receiving the plurality of data to and from the processor 2, a processing of writing or reading the plurality of data to or from the involatile memory 92 and the like, which includes a one chip microcomputer built with ROM, RAM, watchdog timer (WDT); a serial communication controller (SIO); a parallel communication controller (PIO); a counter (CTC) and the like; a selector (SEL) 94 for switching transmission/reception by controlling of CPU 93 for executing the serial communication; a power source voltage detector 95 for monitoring power source voltage for preventing erroneous operation of CPU 93 and the involatile memory 92; the release switch 1b; the connector 1a and so on.

FIG. 13 is a flowchart showing the operation of the endoscope system according to the embodiment.

At step S51, when power source of the processor 2 is inputted, power source is supplied from the CCD drive power source 20 in the processor 2 to CCD 4 in the endoscope 1 via a cable (not explicitly shown) connecting the connector la and the connector 2a. The supply voltage is converted into the desired voltage by the regulator 91 and at step S52, supplied to IC of the involatile memory 92 or the like to start the IC.

Further, at step S53, CPU 6 in the processor 2 transmits endoscope data read command to CPU 93 in the endoscope 1 with regard to the endoscope 1 to provide various data related to the connected endoscope 1 such as the type of endoscope, ID of the endoscope and so on as shown in Table 4.

TABLE 4

| Endoscope data | Content |
|---|---|
| Scope type | Data indicating type of endoscope |
| Cleaning tube | Data related to cleaning tube adapter |
| adapter | mounted before cleaning |
| CCD type | Data indicating type of CCD |
| Scope ID | Data indicating ID (serial number) of endoscope |
| Count | Count data indicating number of time of |
| | use of endoscope |
| Reprocess | Data indicating time of completing |
| completion time | reprocess (used also for |
| | determining whether reprocess is completed) |
| Cleaning | Content data of set cleaning instruction |
| instruction content | |
| Auto leakage | Data indicating result of auto leakage test |
| test result | |
| Name of reprocess | Data indicating executor of reprocess |
| executor | |
| First use | Data indicating first use date |
| Name of facility | Date indicating name of facility |
| Repair record | Data indicating past repair history |
| Check record | Data indicating past check history |
| User inputted data | Arbitrary data inputted by user |

At step S54, when CPU 93 in the endoscope 1 confirms reception of the endoscope data read command, CPU 93 transmits the endoscope data to the processor 2. The processor 2 stores the received endoscope data in RAM 11 or ROM 10.

Further, when “Scope Info.” key (F8) on the keyboard 8 is depressed at step S55, at step S56, the processor 2 displays the endoscope data received at step S54 on the monitor 23.

FIG. 14 shows the monitor 24 displayed with the endoscope data, notation 101 designates endoscope image and notation 102 designates displayed endoscope data. The endoscope data displayed on the monitor 24 may automatically be erased after a pre-selected time period or may be erased by key input on the operation panel 9 or the keyboard 8. Further, with regard to the endoscope data displayed on the monitor 24, the total or a portion of the plurality of data shown in Table 4 may be displayed. FIG. 14 shows an example in which the following four data in the various endoscope data enumerated in Table 4 are displayed on the monitor:
Scope type;
CCD type;
Scope ID; and
First use.

Further, all or a portion of the processings in step S52 through step S55 may automatically be carried out: after inputting the power source of the processor 2 (step S51) as shown by FIG. 13; after depressing the “Scope Info.” key (F8) on the keyboard 8 (step S56); or by depressing an exclusive key on the keyboard 8 or on the operation panel 9.

After step S52, the endoscope data stored in RAM 11 or ROM 10 may be held as it is by storing the endoscope data to ROM 10 even after cutting power supply of the processor 2, or may not be held therein.

Further, in inputting power source of the processor 2 (step S51), the endoscope 1 and the processor 2 may have been already connected, or the endoscope 1 may be connected thereto after inputting power source of the processor 2. In the former case, step S52 through step S56 are carried out as described above, and in the latter case, after supplying voltage to the endoscope 1 by the CCD drive power source 20 in the processor 2, step S52 through step S56 are carried out. After connecting the endoscope 1 and the processor 2, voltage may be supplied immediately to the endoscope 1, or operation of supplying voltage to the endoscope 1 may be carried out by an arbitrary inputting means in the endoscope system such as an arbitrary switch on the keyboard 8, an arbitrary switch on the operation panel 9 or the like. Further, the processing in the latter case may similarly be applied even when the endoscope 1 which is being connected, is detached in starting the processor 2 or may be interchanged by another of the endoscope 1.

This way, according to the embodiment, the processing of transferring various information of the endoscope 1 to the processor 2 and displaying the endoscope information on the monitor 24, can be realized only by depressing exclusive keys on the keyboard 8 and accordingly, information related to the endoscope 1 can be confirmed extremely easily.

Further, according to the above-described first through third embodiments, the keys 33 in the keyboard 8 may be provided in the operation panel 9 of the processor 2 instead of in the keyboard 8. Further, functions of the switches 47, 48 and 50 in keys 33 may be set on a system setup screen.

It is apparent that different embodiments can be constituted in a wide range without deviating from spirit and range of the invention. The invention is not restricted by specific embodiments thereof other than being limited by attached claims.

1. An endoscope system comprising:
an endoscope for taking an image of inside a colon;
a processor for signal processing a taken image from said endoscope;
a filing apparatus for recording and controlling an endoscope image and endoscope related information outputted from said processor; and
a keyboard connected to said processor and having operation keys for inputting said endoscope related information and control keys for controlling said processor and said filing apparatus;
selecting switches for selecting either one of said processor and said filing apparatus; and
a selecting part provided in said processor for selecting a portion or all of said operation keys and said control keys in said keyboard for common use in said processor and said filing apparatus by selecting either one of said processor and said filing apparatus to make it possible to transmit and receive signals based on inputs selected by said selecting switches.

2. The endoscope system according to claim 1,
wherein said keyboard is provided with said selecting switches.

3. The endoscope system according to claim 1, wherein said processor is provided with said selecting switches.

4. The endoscope system according to claim 1, further comprising:
a printer for printing at least the endoscope image or the endoscope related information outputted from the processor or the filing apparatus;
wherein the keyboard or the processor includes setting means that sets a number of the endoscope images printed on one sheet of print paper and a number of print sheets of the printer.

5. The endoscope system according to claim 1, wherein the selecting part for selecting a portion or all of the operation keys is separable from at least one of the processor and the keyboard.

6. The endoscope system according to claim 1, wherein the keyboard includes switches that enable reading a single or a plurality of information related to the endoscope stored in a memory in the endoscope.

7. An endoscope system comprising:
an endoscope and a plurality of endoscope related information apparatuses for processing endoscope related information;
an inputting part for executing an inputting operation for controlling the endoscope and the plurality of endoscope related information apparatuses, the inputting part having a manual inputting part for manually inputting the endoscope related information;
a select inputting part for selecting the manual inputting part and for selecting an apparatus controlled by an input from the inputting part from among the endoscope and the plurality of endoscope related information apparatuses;
a system controlling part for selecting the apparatus controlled by the input from the inputting part by receiving a signal from the selective inputting part;
wherein an output of the selected apparatus is provided to at least two of the endoscope related information apparatuses under control of the system controlling part, whereby the at least two of the endoscope related information apparatuses can process the output based on the inputting operation.

8. The endoscope system according to claim 1, wherein
an endoscope for taking an image of inside a colon;
processor means for signal processing a taken image from said endoscope;
a filing means for recording and controlling an endoscope image and endoscope related information outputted from said processor means; and
a keyboard means connected to said processor means and having operation keys for inputting said endoscope related information and control keys for controlling said processor means and said filing means;
selecting switches means for selecting either one of said processor means and said filing means; and
selecting part means provided in said processor means for selecting a portion or all of said operation keys and said control keys in said keyboard for common use in said processor means and said filing means by selecting either one of said processor means and said filing means to make it possible to transmit and receive signals based on inputs selected by said selecting switches means.

9. The endoscope system according to claim 8,
wherein said keyboard means is provided with said selecting switches means.

10. The endoscope system according to claim 8, wherein said processor means is provided with said selecting switches means.

11. The endoscope system according to claim 8, further comprising:
a printer for printing at least the endoscope image or the endoscope related information outputted from the processor means or the filing means;
wherein the keyboard means or the processor means includes setting means for setting a number of the endoscope images printed on one sheet of print paper and setting a number of print sheets of the printer.

12. The endoscope system according to claim 8, wherein the selecting part means for selecting a portion or all of the operation keys is separable from at least one of the processor means and the keyboard means.

13. The endoscope system according to claim 8, wherein the keyboard means includes switches that enable reading a single or a plurality of information related to the endoscope stored in a memory means in the endoscope.

14. The endoscope system according to claim 13, comprising a memory part for holding a set condition set by said selecting switches means and said selecting part means.

15. A method of handling an endoscope system, the method comprising the steps of:
using an endoscope for taking an image of inside a colon;
subjecting a taken image from said endoscope to signal processing using a processor;
recording and controlling an endoscope image and endoscope related information outputted from said processor, including using a filing apparatus;
inputting said endoscope related information and control keys for controlling said processor and said filing apparatus by operating a keyboard connected to said processor and using operation keys;
selecting either one of said processor and said filing apparatus by operating selecting switches; and
selecting a portion or all of said operation keys and said control keys in said keyboard for common use in said processor and said filing apparatus by selecting either one of said processor and said filing apparatus to make it possible to transmit and receive signals based on inputs selected by said selecting switches.
